(19) United States
(12) Patent Application Publication
(10) Pub. No.: US 2025/0273319 A1
(43) Pub. Date: Aug. 28, 2025

(54) Artificial Intelligence Based Method And Application For Assessing Fitness, Blood Glucose Levels And Calorie Consumption With Real Time Feedback

(71) Applicant: Syed Ahad Ali, Chantilly, VA (US)

(72) Inventor: Syed Ahad Ali, Chantilly, VA (US)

(21) Appl. No.: 19/004,365

(22) Filed: Dec. 29, 2024

Related U.S. Application Data
(60) Provisional application No. 63/616,505, filed on Dec. 29, 2023.

(51) Int. Cl. G16H 20/30; G06Q 30/0601; G06Q 50/00; G06V 10/82; G06V 20/62; G06V 20/68; G10L 15/16; G10L 15/22; G16H 20/60

(52) U.S. Cl. CPC G16H 20/30; G06Q 30/0631; G06Q 50/01; G06V 10/82; G06V 20/63; G06V 20/68; G10L 15/16; G10L 15/22; G16H 20/60; G10L 2015/223

(57) ABSTRACT

The invention is an AI-powered fitness and health management system providing personalized, dynamic recommendations tailored to user-specific goals such as weight loss, muscle gain, and diabetes management. Leveraging Artificial Neural Networks (ANN), Decision Tree algorithms (J48), and Convolutional Neural Networks (CNN), the system analyzes user inputs, including voice commands, wearable device data, blood glucose readings, and food scans, to deliver real-time, adaptive meal and workout plans. Voice activation, initiated with the command “Run Fitzy”, enables hands-free logging of meals, workouts, and health metrics. Computer vision technology scans food labels and meal images, extracting nutritional data and automatically integrating it into the user's profile. The system dynamically adjusts remaining daily meals and activities to meet caloric, fitness, and glucose targets. Social networking features include geo-tagging, fitness challenges, and monetizable content sharing. Built on a secure, scalable cloud infrastructure, the platform integrates e-commerce functionality and ensures compliance with data protection standards.

This non-provisional patent application is filed while asserting priority based on the U.S. application No. 63/616,505.

### TECHNICAL FIELD OF INVENTION

The present invention relates to the fields of health, fitness, artificial intelligence, wearable technology, and social networking. More specifically, it pertains to the development of a comprehensive fitness and health management application that leverages machine learning, voice recognition, and computer vision technologies to provide personalized health and fitness recommendations, real-time data analysis, and social connectivity. The application features voice-activated functionalities for food intake logging, fitness tracking, and blood glucose monitoring, enabling seamless and user-friendly input and command execution. It includes systems for real-time blood glucose monitoring and analysis, supported by AI-driven feedback to optimize dietary and fitness plans. The invention enables users to create detailed profiles for tracking progress and sharing milestones, and incorporates social media features such as geo-tagging, community fitness challenges, and connections with others in shared fitness spaces, such as gyms. Additionally, it provides an integrated e-commerce platform for buying and selling health and fitness-related products. The invention supports wearable device synchronization and is built on a secure, cloud-based infrastructure for scalable, real-time operation.

### BACKGROUND OF THE INVENTION

### Overview

Health and fitness management has become increasingly reliant on technology, with the proliferation of fitness tracking applications and wearable devices. Existing solutions often provide users with the ability to log activities, track workouts, and monitor basic health metrics. However, these applications typically lack the capacity to deliver truly personalized and adaptive recommendations in real time based on dynamic user data. Furthermore, the integration of voice-activated functionalities, real-time glucose monitoring, and comprehensive social connectivity features remains underdeveloped in current systems.

Conventional applications also fail to offer a seamless user experience that combines nutrition tracking, fitness recommendations, and social interaction in a single platform. Users are often required to switch between multiple applications for different functionalities, such as tracking meals, monitoring blood glucose levels, and connecting with other fitness enthusiasts or professionals.

Moreover, while many fitness applications allow for data input and tracking, they often depend on manual entry, which can be cumbersome and prone to errors. Few systems provide robust voice-recognition capabilities to simplify user interaction. Similarly, while e-commerce platforms for health and fitness products exist, they are rarely integrated within fitness applications, limiting the user's ability to engage in a holistic health and wellness ecosystem.

### Need for the Invention

There is a need for a comprehensive fitness application that combines advanced artificial intelligence with voice-activated functionalities, wearable device integration, and real-time health monitoring. Such a system should enable users to log activities effortlessly, receive personalized guidance, and interact socially with others in their fitness communities. The ability to monitor blood glucose levels in real time and adjust dietary and fitness recommendations dynamically is particularly important for users managing chronic conditions such as diabetes.

Additionally, the integration of social networking features, including geo-tagging and user connectivity within specific locations such as gyms, addresses the growing demand for community-based fitness solutions. A unified platform that also supports virtual stores for selling health and fitness products can further enhance user engagement and create a comprehensive ecosystem for health and wellness.

The present invention addresses these needs by providing an all-encompassing fitness application with advanced AI capabilities, voice-driven interactions, real-time health monitoring, and social networking features, ensuring a seamless and user-centric experience.

### PATENT CLASSIFICATION

### Cooperative Patent Classifications (CPC)

a. G06N 20/00—Machine learning
   1. Covers the use of AI and machine learning algorithms for real-time personalized recommendations, adaptive workout plans, and blood glucose monitoring
b. G10L 15/22—Speech or voice recognition
   1. Specifically relevant to the app's voice-activated functionalities for food intake, fitness tracking, and blood glucose monitoring.
c. A61B 5/00—Measuring for diagnostic purposes
   1. Covers health monitoring functionalities such as tracking fitness parameters and blood glucose levels.
d. A61B 5/145-Measuring characteristics of blood, e.g., glucose levels
   1. Relevant to the real-time blood glucose monitoring with AI-driven feedback.
e. A61B 5/11-Measuring physical parameters of the body
   1. Encompasses fitness tracking (e.g., heart rate, activity monitoring) through wearable devices.
f. A63B 24/00-Equipment for physical training or training methods
   1. Relevant to AI-optimized workout routines and fitness tracking features.
g. G06Q 50/02-Systems or methods for health care management
   1. Related to the overall health and fitness management capabilities of the app.
h. G06Q 30/06-Commerce, e.g., shopping or e-commerce
   1. Relevant for the app's feature allowing users to create stores for health and fitness item sales.
i. H04W 4/021-Services specially adapted for wireless communication networks 1. Covers geo-tagging, location tracking, and interactions with other users within the same gym.

### US CLASSIFICATIONS

a. Class 704—Data Processing: Speech Signal Processing, Linguistics, Language Translation, and Audio Compression/Decompression
   1. Specifically applicable to the voice recognition capabilities for food intake logging, fitness tracking, and blood glucose monitoring.
b. Class 706—Data Processing: Artificial Intelligence
   1. Relevant to the app's AI-driven features such as machine learning algorithms for personalized health recommendations and adaptive plans.
c. Class 382—Image Analysis
   1. Pertinent to the app's computer vision functionalities for analyzing food labels to extract nutritional information.
d. Class 709—Electrical Computers and Digital Processing Systems: Multicomputer Data Transferring
   1. Could apply to the synchronization of data across devices and the integration with wearable technologies.
e. Class 705—Data Processing: Financial, Business Practice, Management, or Cost/Price Determination
   1. Applicable to the e-commerce component, where users can create stores to sell health and fitness items.

### Patent Subject Matter

a. Speech recognition
b. Artificial intelligence
c. Machine learning
d. Fitness tracking
e. Social media
f. Real-time feedback
g. Remote monitoring
h. Personalized health recommendations
i. Personalized nutrition recommendations
j. E-commerce marketplace for health and fitness

Intended Audience: The invention is intended for a broad spectrum of users who seek to manage and improve their health and fitness through advanced technological solutions. The primary audience includes fitness enthusiasts, health-conscious individuals, and those managing chronic conditions such as diabetes, who will benefit from the application's real-time monitoring, personalized guidance, and fitness optimization features. The invention is also designed for individuals utilizing wearable fitness technology, providing seamless integration and consolidated tracking of health metrics.

In addition to individual users, the invention is intended for entities and professionals within the fitness and health industries. This includes personal trainers, nutritionists, and health coaches, who can leverage the application to monitor client progress and provide tailored recommendations. Furthermore, the invention serves fitness centers and gyms by enabling member engagement through social networking features, such as geo-tagging and user connection within shared facilities.

The application also addresses the needs of entrepreneurs and small businesses, providing a platform to create virtual stores for selling health and fitness-related products. Corporate wellness programs and healthcare providers may also integrate the invention into their operations to promote health and wellness among employees and patients, respectively.

By targeting individual users, professionals, and institutional entities, the invention targets a diverse audience seeking innovative solutions for fitness and health management.

### Relevant Research and Studies

The present invention builds upon advancements in artificial intelligence, voice recognition, and health monitoring technologies, as well as studies highlighting the importance of social engagement in fitness adherence. Key findings in the field include:

1. Smith et al. (2020): Demonstrated that machine learning algorithms can provide personalized dietary and fitness recommendations by analyzing real-time health data, significantly improving user outcomes.

2. Johnson et al. (2019): Highlighted the effectiveness of real-time blood glucose monitoring in managing chronic conditions such as diabetes and its role in tailoring dietary advice.

3. Taylor et al. (2021): Explored the impact of social networking and community engagement on fitness adherence, emphasizing the value of location-based social interactions.

4. Miller et al. (2022): Showed that voice-activated technology enhances user engagement and accessibility in health applications, particularly by simplifying input processes for tracking and monitoring.

These studies underscore the need for an integrated platform combining AI-driven real-time monitoring, voice-activated interaction, and social networking features. The present invention addresses these needs, offering a unified solution that improves user experience and adherence to health and fitness goals.

### BRIEF SUMMARY OF INVENTION

The present invention relates to an artificial intelligence (AI)-driven system designed to deliver personalized and adaptive speech therapy and language learning solutions. The system utilizes AI technologies, including speech recognition, machine learning, and facial recognition, to analyze user speech patterns, diagnose communication disorders, and provide real-time feedback through customized therapy sessions. It is aimed at addressing a wide range of speech disorders, including stammering, articulation issues, voice disorders, receptive and expressive language disorders, and accent modification. Additionally, it offers support for individuals recovering from speech impairments caused by medical conditions such as strokes or traumatic brain injuries.

The invention offers several key advantages:

1. Personalization: The AI system tailors therapy sessions to each user's unique speech patterns and progress. The exercises are continuously adjusted in complexity to promote steady improvement, providing individualized care that is traditionally challenging in group-based or remote settings.

2. Accessibility: By offering a fully virtual platform, the invention ensures access to high-quality speech therapy services for individuals in remote or underserved areas, or for those facing financial barriers to traditional therapy. This accessibility helps to close the gap between the need for speech therapy and the availability of resources.

3. Engagement and Gamification: The platform integrates gamified elements, such as interactive exercises and visual reinforcement, to enhance user engagement. This is especially beneficial for children and users who may find traditional therapy tedious or intimidating.

4. Real-time Feedback and Continuous Monitoring: The system provides immediate feedback on speech and articulation, simulating real-time interactions with a speech therapist. In addition, it allows for remote progress monitoring, enabling speech-language pathologists and caregivers to track improvement and adjust therapy plans as needed, without requiring in-person sessions.

5. Wide Applicability: The invention supports a diverse audience, including children with developmental delays, adults seeking accent reduction, stroke survivors, and individuals with cognitive impairments. It also provides flexibility for self-learners or individuals seeking to improve communication skills for professional development.

6. Cost-effectiveness: By providing a virtual and self-paced alternative to traditional in-person speech therapy, the invention offers a more cost-effective solution, reducing the need for frequent clinic visits while ensuring continuous progress through AI-driven assessments and feedback.

Overall, the invention bridges the gap between accessibility and the need for effective, high-quality speech therapy, leveraging AI to create an innovative and adaptive system that empowers users to overcome communication barriers.

### Summary of Invention

The present invention is an AI-powered fitness application that delivers a comprehensive, personalized solution for health and fitness management. By leveraging advanced artificial intelligence, including Artificial Neural Networks (ANN), Decision Tree algorithms such as J48, and Convolutional Neural Networks (CNN), the invention transforms how users monitor their health, achieve fitness goals, and engage with fitness communities.

The system architecture integrates multiple components, including a user interface layer for seamless interaction, an AI model layer powered by ANN, J48, and CNN for intelligent decision-making, and a cloud-based infrastructure for secure data storage and real-time synchronization. Voice-activated functionality, initiated with the command “Run Fitzy”, enables users to log food intake, track fitness activities, and monitor health metrics, including real-time blood glucose levels. These inputs are processed dynamically, allowing the AI to deliver adaptive recommendations tailored to individual goals such as weight loss, muscle gain, and diabetes management.

The ANN processes complex, non-linear relationships between user health data and goals, while the J48 decision tree categorizes and generates rule-based outputs for optimal dietary and workout plans. The CNN powers food scanning and image recognition, extracting nutritional information from labels and meal images and seamlessly integrating it into the user's profile. The system dynamically adjusts remaining daily meals and fitness routines to align with caloric, fitness, and glucose targets.

Wearable device integration enables real-time tracking of health metrics such as heart rate, steps, and sleep patterns. AI-enhanced workout planning employs muscle confusion techniques to create diverse and effective routines that prevent fitness plateaus. The invention also provides gamification features, including activity streaks, rewards, and progress visualizations, to enhance user engagement and retention.

Social networking capabilities include geo-tagging, collaborative fitness challenges, and user-generated content sharing. The platform fosters community engagement by connecting users with others in shared fitness environments such as gyms and enabling monetization of high-quality fitness content through ad revenue-sharing. An integrated e-commerce marketplace allows users to buy and sell health and fitness-related products, supported by AI-driven recommendations and secure payment processing.

The backend system employs PostgreSQL for data management and a scalable cloud-based infrastructure to handle computational demands efficiently. Security measures, including end-to-end encryption and compliance with GDPR, HIPAA, and US data protection regulations, safeguard user information.

By leveraging innovative AI models, computer vision, and voice recognition technologies, the invention addresses key limitations in prior art such as MyFitnessPal and Cronometer. It provides a unified, user-centric platform offering real-time AI-driven recommendations, dynamic adjustments, integrated social networking, and e-commerce functionalities. This invention sets a new standard for secure, scalable, and engaging fitness and health management.

### BRIEF DESCRIPTION OF DRAWINGS

The invention will be more readily understood by reference to the following description, taken with the accompanying drawings, in which:

FIG. 1: A diagram illustrating the code architecture of the application, showing the integration of Artificial Neural Networks (ANN), decision tree algorithms (J48), API layers, cloud-based infrastructure, and user interface components.

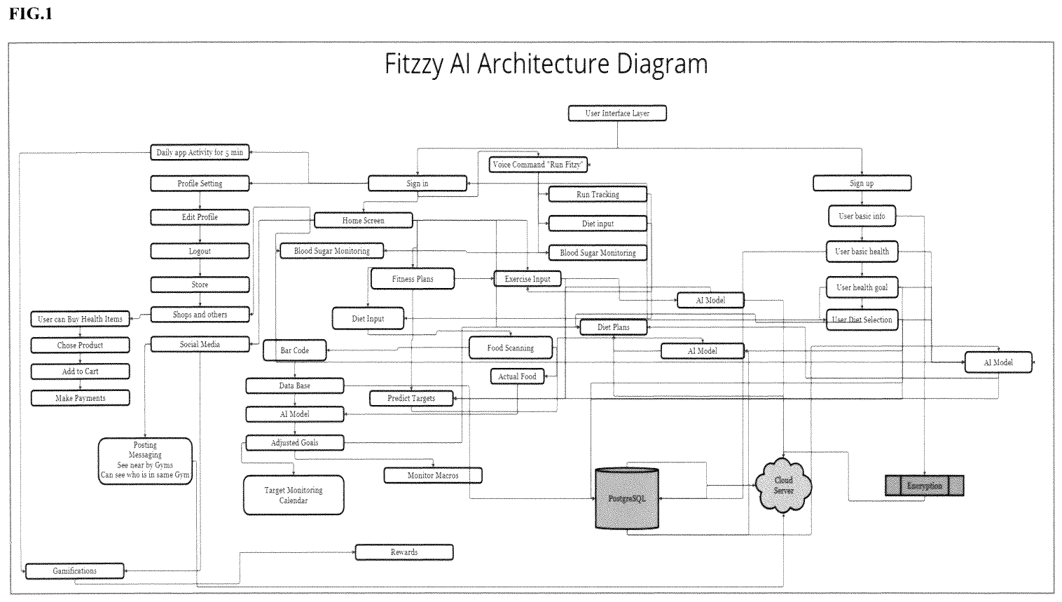

FIG. 2: A flowchart depicting the operational workflow of the application, highlighting the voice command functionality for food intake logging, fitness tracking, and health monitoring.

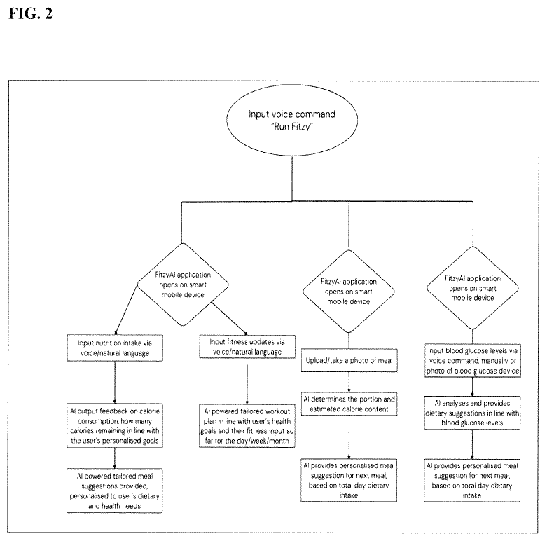

FIG. 3: Screenshots of the application setup screens for user profile creation, displaying the initial steps in onboarding users.

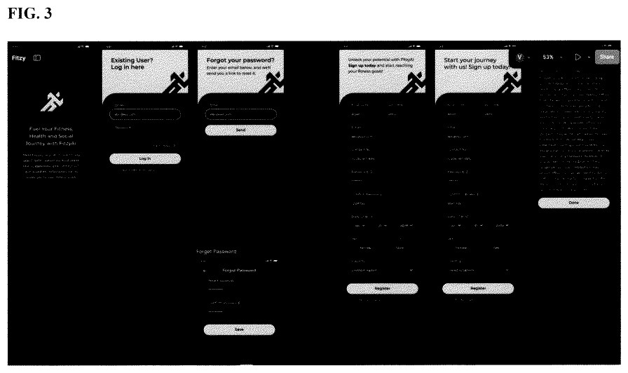

FIG. 4: Screenshots showcasing additional profile setup screens, where users input personal details such as age, weight, height, and activity levels.

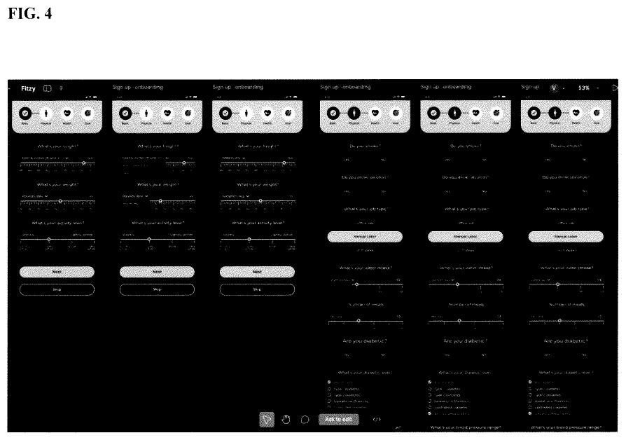

FIG. 5: A screenshot of the profile setup process asking users whether they have diabetes and specifying the type of diabetes, if applicable.

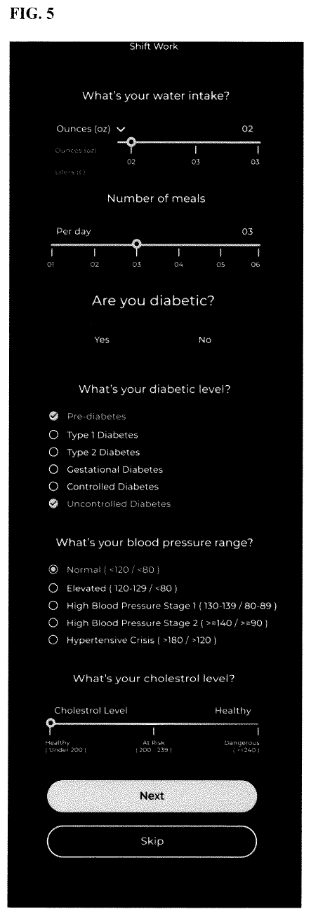

FIG. 6: Screenshots illustrating further profile setup options related to fitness goals and nutritional preferences.

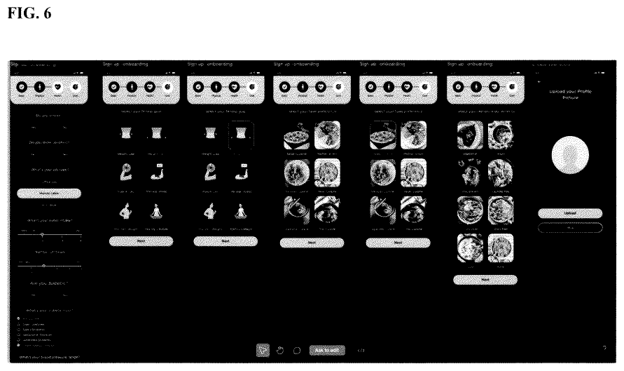

FIG. 7: Screenshots demonstrating how the AI adapts calorie recommendations dynamically based on the user's nutritional intake.

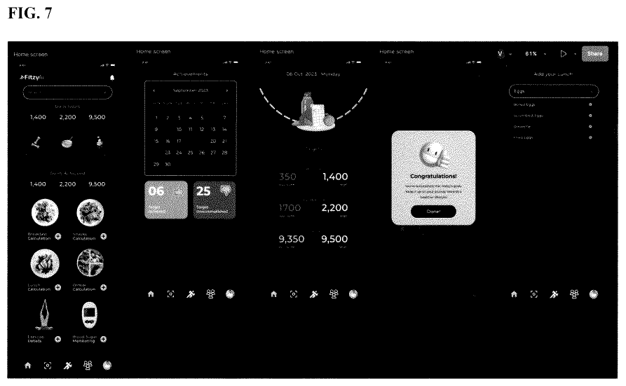

FIG. 8: Screenshots of the fitness streak tracking feature, displaying user progress and motivational elements.

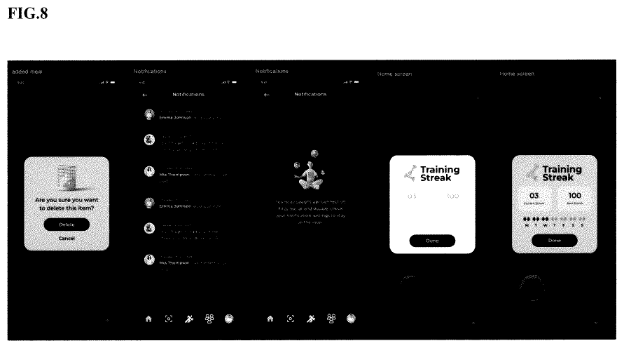

FIG. 9: Screenshots showing AI-adapted fitness plans and real-time blood glucose monitoring interfaces.

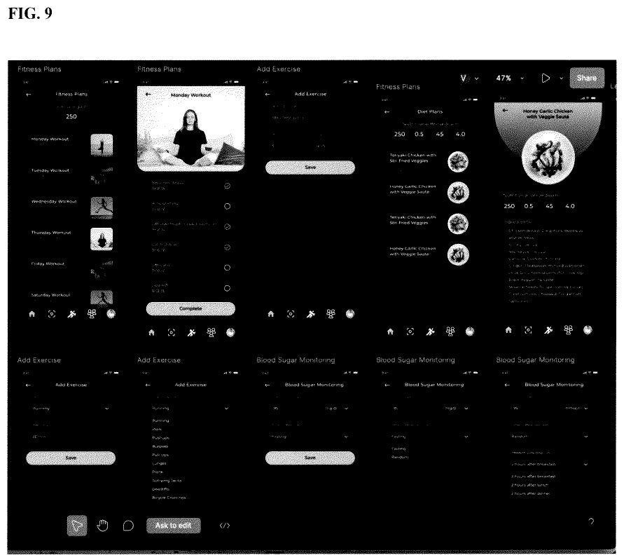

FIG. 10: Screenshots illustrating the manual input functionality for health metrics such as blood glucose levels and other fitness data.

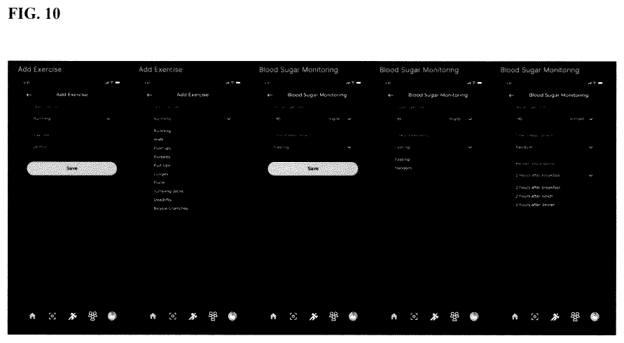

FIG. 11: Screenshots of the integrated e-commerce store, enabling users to buy and sell health and fitness products.

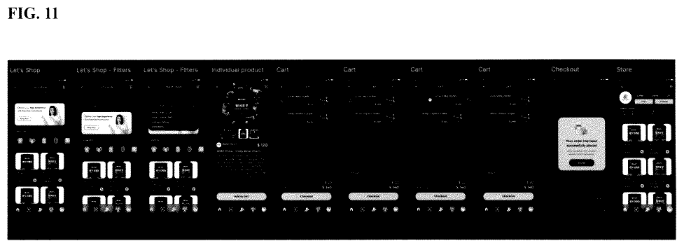

FIG. 12: Depictions of the food scanning technology, showing how users can scan food labels or images to extract nutritional data using computer vision.

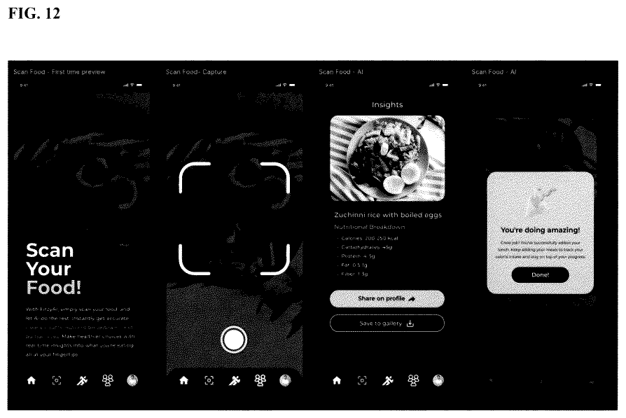

FIG. 13: Screenshots highlighting the voice command feature for inputting data and the corresponding AI-generated outputs for dietary and fitness recommendations.

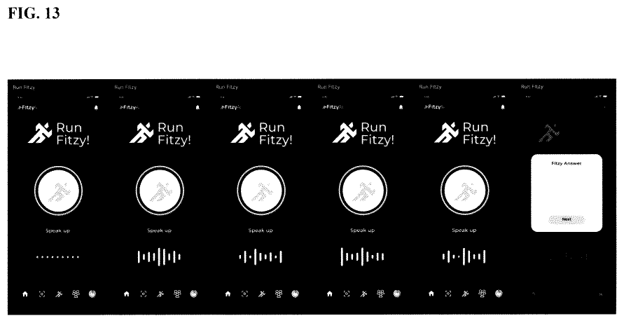

FIG. 14: Screenshots of the social media profile feature, allowing users to share progress, connect with others, and engage in community challenges.

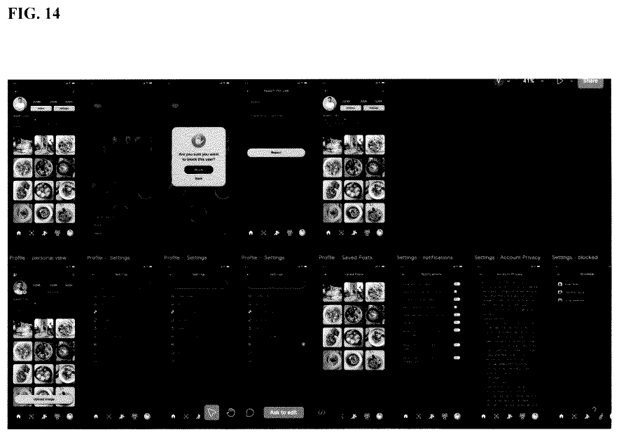

FIG. 15: Entity relationship diagram of the system

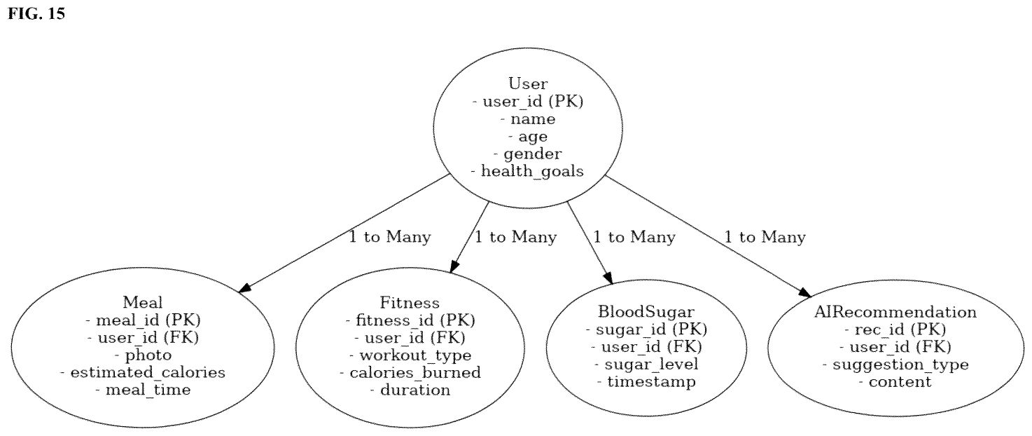

### DETAILED DESCRIPTION OF INVENTION

The present invention is an AI-powered fitness application that provides a comprehensive solution for health and fitness management. By integrating advanced artificial intelligence, including Artificial Neural Networks (ANN), Decision Tree algorithms (J48), voice recognition, and computer vision technologies, the invention offers dynamic and personalized recommendations to users. The application features a robust technical architecture, multi-functional capabilities, and compliance with GDPR, HIPAA, and other US data protection regulations, ensuring secure and scalable operations.

### Technical Architecture

### 1. AI Algorithms:

Artificial Neural Networks (ANN): Processes complex, non-linear relationships between health parameters, historical data, and user goals. ANN models enable precise, adaptive recommendations for nutrition, fitness, and health management.
Decision Tree Algorithm (J48): Rule-based classification for generating dietary adjustments or fitness plans based on user inputs and predefined logic.
Convolutional Neural Networks (CNN): Enables image recognition for food label scanning and meal image analysis.
Training Datasets:
   A database of thousands of foods and meals with detailed caloric and nutritional breakdowns.
   Comprehensive fitness routines covering running, strength training, cardio, and flexibility exercises.
   User activity data reflecting goals like weight loss, muscle gain, diabetes management, and behavioral patterns.
Continuous Learning: User feedback loops refine predictions and recommendations over time, improving accuracy.

### 2. Voice Recognition and Natural Language Processing (NLP):

Activation Command: Voice functionality is initiated with “Run Fitzy”, enabling hands-free interaction.
   Example Commands:
      “Run Fitzy, log my lunch” to record food intake.
      “Run Fitzy, track my workout” for fitness activity tracking.
      “Run Fitzy, record my blood sugar” to input health metrics.
NLP Functionality: Ensures accurate recognition and interpretation of diverse voice commands, adapting to user speech patterns and preferences.

### 3. Cloud-Based Infrastructure:

Employs a scalable backend for real-time synchronization, secure storage, and high computational efficiency.
PostgreSQL databases manage structured data, enabling quick retrieval and scalability for millions of users.

### 4. Data Security:

Ensures data encryption in transit and at rest.
Regular audits ensure compliance with GDPR, HIPAA, and other relevant regulations for health data security.

### Key Functionalities

### 1. Dynamic Calorie and Nutritional Adaptation:

AI adjusts calorie and nutrient recommendations dynamically based on food intake, exercise, and historical trends.
   Example:
      If a user logs a high-calorie meal, the system compensates by recommending lower-calorie options for subsequent meals.
      If protein intake is insufficient, the system suggests protein-rich foods.
Adjustments are informed by extensive datasets and refined through continuous learning.

### 2. Profile Customization:

Users input age, weight, height, activity levels, and health conditions (e.g., pre-diabetes, Type 1, Type 2).
Personalized plans are generated based on this information and updated dynamically.

### 3. Fitness and Health Monitoring:

Wearable integration allows real-time tracking of steps, heart rate, and sleep patterns.
Blood glucose monitoring through manual input or device integration provides actionable dietary adjustments for glycemic control.

### 4. AI-Enhanced Workouts:

Dynamically adjusts workout intensity, type, and duration based on performance metrics.
Prevents fitness plateaus using muscle confusion principles.
Suggests recovery exercises and rest days to optimize performance.

### 5. Food Scanning and Image Recognition:

Food Label Scanning: Deciphers calorie content, macronutrients, and micronutrients from barcodes or labels.
Meal Image Analysis: Identifies food items and estimates portion sizes for accurate caloric and nutrient calculations.
Integration into User Profiles:
   Updates daily logs with nutritional data.
   Dynamically adjusts meal plans to meet caloric and dietary goals.

### 6. Social Media and Community Engagement:

Personalized Social Profiles: Users track fitness achievements and share updates.
Geo-Tagging and Challenges: Facilitates connections within fitness spaces, promoting collaboration and competition.
Ad Revenue Sharing: Allows users to monetize high-quality fitness content.
Gamification Features: Includes leaderboards, badges, and rewards for fitness streaks.

### 7. E-Commerce Integration:

Enables users to buy and sell health and fitness-related products.
Features AI-driven price comparison and secure payment processing.

### Operational Workflow

### 1. Setup and Onboarding:

Users input demographic, health, and fitness data during profile creation.
The system generates tailored recommendations based on input parameters.

### 2. Real-Time Data Integration:

Processes wearable metrics, voice commands, and manual inputs dynamically.
Updates fitness and nutritional plans in real time.

### 3. Adaptive AI Recommendations:

AI models continuously refine recommendations based on user behavior and feedback.
Example Adjustments:
   After a workout, the system suggests recovery meals based on burned calories.

### 4. Progress Visualization:

Displays user trends in calorie intake, fitness metrics, and health goals through intuitive dashboards.

### Example Use Cases

### 1. Nutritional Adjustment:

A user logs a high-carb breakfast via “Run Fitzy, log my breakfast of pancakes.”
   The system adjusts lunch and dinner to be lower in carbohydrates, maintaining the user's daily caloric target.

### 2. Workout Tracking and Suggestions:

A user tracks a run using “Run Fitzy, track my run.”
   The system logs distance, pace, and calories burned, and recommends post-run stretches.

### 3. Community Engagement:

Users participate in geo-tagged challenges within their gym, competing on step counts for rewards.

### 4. Food Scanning:

A user scans a protein bar barcode, and the system updates their daily log, recommending complementary meals.

### Innovations and Benefits

### 1. Personalized AI Integration:

Combines ANN, J48, and CNN for precise, real-time adaptations.

### 2. Dynamic Adjustments:

Ensures recommendations remain relevant as user inputs change.

### 3. Holistic User Experience:

Blends health monitoring, social networking, and e-commerce into a unified platform.

### Prior Art Comparison

Several platforms currently exist in the field of health and fitness tracking, with notable examples including MyFitnessPal and Cronometer. These platforms provide users with tools for logging food intake, monitoring exercise, and tracking basic health metrics. While they have achieved significant popularity and offer valuable functionality, both platforms present substantial limitations that the present invention overcomes by integrating advanced artificial intelligence, voice-activated features, real-time health monitoring, and comprehensive social and e-commerce functionalities.

### MyFitnessPal

MyFitnessPal is one of the most widely used digital solutions for health and fitness management. It includes a large database of food items, allowing users to log their meals manually and calculate caloric and nutritional intake. The platform also integrates with some wearable devices to synchronize activity data such as steps and calories burned. Additionally, it offers a community feature where users can share progress and connect with others.

### Limitations of MyFitnessPal

### 1. Manual Input Dependency:

Users are required to log food intake and activities manually, which is time-consuming and prone to errors. There is no mechanism for effortless, automated tracking.

### 2. Lack of Real-Time AI Adaptation:

MyFitnessPal does not leverage advanced artificial intelligence to analyze real-time user behavior or health data. Recommendations are static and generalized rather than adaptive or dynamic.

### 3. No Voice-Activated Features:

The platform lacks voice recognition capabilities for food logging, fitness tracking, or health monitoring. This limits accessibility for users who prefer hands-free interaction.

### 4. Limited Health Monitoring:

While MyFitnessPal tracks calories and exercise metrics, it does not offer real-time monitoring of critical health metrics, such as blood glucose levels, nor does it provide AI-driven adjustments based on these metrics.

### 5. Basic Social Networking:

MyFitnessPal allows users to connect with others but lacks localized social networking features such as geo-tagging to enable interactions with people in shared physical environments like gyms.

### 6. No E-Commerce Integration:

Users cannot create virtual stores or engage in buying and selling health and fitness products, limiting its scope as a holistic ecosystem.

### Cronometer

Cronometer is another prominent application in the field, known for its focus on detailed nutrient tracking. It provides users with tools to analyze their vitamin and mineral intake, emphasizing micronutrient management alongside caloric tracking. Cronometer also integrates with wearable devices to log activity data.

### Limitations of Cronometer

### 1. Manual Logging Requirements:

Like MyFitnessPal, Cronometer relies heavily on manual entry for food logging and fitness tracking, which can be cumbersome and inefficient for users.

### 2. No Voice-Activated Features:

The platform does not offer voice recognition functionality, which would greatly enhance usability and reduce the effort required for data entry.

### 3. Limited Real-Time Adaptation:

Although Cronometer provides detailed insights into nutrient intake, it does not adapt dynamically to real-time health data such as blood glucose levels.

### 4. Minimal Social Networking:

Cronometer lacks robust community-building features. There are no tools to connect users based on location or shared fitness environments.

### 5. No Integrated E-Commerce Features:

The platform does not facilitate the buying or selling of health and fitness products, reducing its utility for users seeking a comprehensive fitness ecosystem.

### Advantages of the Present Invention Over Prior Art

The present invention addresses these limitations by offering a unified platform that integrates the strengths of existing systems while introducing novel features that significantly enhance usability, adaptability, and functionality.

### 1. AI-Powered Real-Time Recommendations:

Unlike MyFitnessPal and Cronometer, the invention uses advanced artificial intelligence to analyze real-time user data, including blood glucose levels, to provide dynamic, personalized dietary and fitness recommendations.

### 2. Voice-Activated Functionality:

The invention introduces voice recognition technology to simplify food intake logging, fitness tracking, and health monitoring, making it accessible and efficient for users.

### 3. Comprehensive Health Monitoring:

The invention integrates wearable devices to track real-time health metrics, including heart rate and blood glucose levels, and provides AI-driven feedback for immediate adjustments.
   4. Social Networking with Geo-Tagging:
Users can connect with others in their gym or local fitness community through geo-tagging and location-based tracking, fostering a collaborative and engaging experience.

### 5. Integrated E-Commerce Platform:

The invention enables users to create virtual stores for buying and selling health and fitness-related products, creating a unique marketplace within the platform.

### 6. Unified Ecosystem:

By combining advanced AI, voice-activated input, social networking, and e-commerce functionalities, the present invention eliminates the need for users to rely on multiple applications to meet their health and fitness needs.

By addressing the deficiencies of prior art systems such as MyFitnessPal and Cronometer, the present invention establishes a comprehensive, user-centric solution that advances the field of health and fitness management.

### BEST MODE OF CARRYING OUT THE INVENTION

The best mode for carrying out this invention is through a mobile and web application that users can access on devices equipped with cameras, microphones and location tracking. Users interact with AI algorithms and real time feedback, they can easily input their fitness and health information via voice command which quickly provides them with an AI generated output tailored specifically to their goals. This comprehensive solution is accessible, engaging, and tailored for individuals of all ages with varying health and fitness capabilities, comorbidities and goals.

This invention stands apart from existing tools by offering an engaging, holistic, and adaptive approach to speech therapy, designed to provide the highest level of personalized care and support.

### List of AI Models and Backend Services

The invention leverages advanced machine learning models and backend infrastructure to deliver adaptive, personalized, and real-time health and fitness recommendations. These components form the core of the system's intelligent functionalities:

### 1. Artificial Neural Networks (ANN):

Purpose:
   Used for non-linear relationship modeling between diverse user data (e.g., health metrics, dietary habits, activity levels) and fitness or health goals (e.g., weight loss, muscle gain, diabetes management).
Key Functions:
   Dynamic calorie adjustments based on real-time food intake and exercise data.
   Personalized workout recommendations, optimized for individual performance and recovery needs.
   Prediction of nutrient deficiencies or surpluses to ensure balanced meal plans.
Training Data:
   Extensive datasets containing:
      Thousands of food items and meal combinations, including nutritional and caloric breakdowns.
      Fitness data spanning various workout types, intensities, and performance metrics (e.g., strength training, cardio).
      Behavioral patterns and user interaction logs to enhance adaptive AI-driven recommendations.

### 2. Decision Tree Algorithm (J48):

Purpose:
   Enables structured, rule-based decision-making for tasks like tailoring fitness plans and categorizing dietary inputs.
Key Functions:
   Recommending appropriate workout intensity levels based on user history and real-time performance.
   Adjusting meal recommendations for specific health conditions such as diabetes, high cholesterol, or caloric surplus/deficit.
Training Data:
   Historical user interactions, categorized by health profiles and behavioral trends.
Specialized datasets reflecting condition-specific dietary and fitness requirements.

### 3. Natural Language Processing (NLP):

Purpose:
   Powers voice recognition and interpretation, enabling hands-free user interactions.
Key Functions:
   Understanding and executing user commands such as, “Run Fitzy, log my breakfast of oatmeal and banana.”
   Interpreting natural language inputs for accurate logging of food items, health metrics, and fitness activities.
Technical Tools:
   Fine-tuned pre-trained models (e.g., GPT-based systems, TensorFlow NLP libraries) for fitness-related tasks.
   Tokenization, entity extraction (e.g., identifying food types), and contextual analysis for interpreting voice inputs.

### 4. Computer Vision:

Purpose:
   Extracts nutritional information from food labels and meal images for seamless logging and calorie tracking.
Key Functions:
   Detecting food items in meal images using object detection and image segmentation algorithms.
   Estimating portion sizes and calculating caloric values.
Technical Tools:
   Pre-trained convolutional neural networks (CNNs) such as ResNet or YOLO, fine-tuned on food image datasets for accuracy.
   Integration with OpenCV and TensorFlow/Keras for scalable image analysis workflows.

### Backend Services and Data Storage

### 1. Functionality:

The backend infrastructure securely stores, processes, and retrieves user data, supporting real-time operations and ensuring privacy compliance (e.g., HIPAA, GDPR).

### 2. Modules:

User Data Management:
   Stores detailed records of user activities, performance, and personalized recommendations.
Cloud Storage:
   Manages user-generated data, such as food scans, workout logs, and voice command histories.
   Ensures scalability and secure storage of sensitive information, including health metrics and blood glucose readings.

### 3. Key Features:

Real-Time Data Retrieval:
   Retrieves and synchronizes session data, including food logs, exercise results, and blood glucose levels.
Dynamic Updates:
   Supports adaptive changes to meal plans, workout routines, and health recommendations based on new user inputs.
Privacy and Security:
   Implements encryption for data in transit and at rest, ensuring compliance with GDPR and HIPAA regulations.

### 4. Example Tasks:

Retrieving stored session logs to visualize user progress and adjust fitness or health plans dynamically.
· Storing assessment results to refine future dietary or exercise recommendations.

### Programming Languages

The invention leverages a combination of programming languages and frameworks tailored to its diverse technical and functional requirements:

### 1. Python:

Purpose:
   AI model development, training, and deployment.
Libraries Used:
   TensorFlow and Keras: For implementing Artificial Neural Networks (ANN) and Convolutional Neural Networks (CNN) for tasks like food image recognition and calorie estimation.
   Scikit-learn: For decision trees (J48) and auxiliary machine learning tasks such as data preprocessing and feature selection.
   NLTK and SpaCy: For Natural Language Processing (NLP), enabling voice command recognition and execution.

### 2. JavaScript:

Purpose:
   Front-end development for creating an interactive and user-friendly interface (UI).
Frameworks Used:
   React.js: For developing dynamic, responsive UI components and enabling seamless user interactions.

### 3. Java:

Purpose:
   Backend functionality, business logic, and API integration for wearable devices and external services.
Frameworks Used:
   Spring Boot: For scalable and modular microservices, ensuring efficient backend operations and API management.

### 4. SQL:

Purpose:
   Managing structured data storage, retrieval, and real-time updates in the PostgreSQL database.
Database Design:
   Relational tables for user profiles, food datasets, fitness logs, blood glucose readings, and wearable device metrics.

### 5. Swift and Kotlin:

Purpose:
   Native mobile app development for iOS and Android platforms.
Key Features Implemented:
   GPS Tracking: For fitness activity tracking and geo-tagging in social networking features.
   Voice Activation: Integration with platform-specific APIs (e.g., SiriKit for iOS, Google Assistant for Android) to enable voice commands like “Run Fitzy, track my run.”

### System Architecture

The system employs a multi-layered architecture designed for scalability, reliability, and real-time responsiveness, ensuring seamless user experience and efficient backend operations:

### 1. Frontend Layer:

Technologies Used:
   React.js for developing web-based user interfaces.
   Swift/Kotlin for building native mobile applications for iOS and Android.
Features Implemented:
   Voice Command Interaction: Enables hands-free logging of meals, workouts, and health metrics.
   Progress Visualization Dashboards: Provides intuitive charts and summaries for tracking fitness and nutritional goals.
   Social Media and Marketplace Modules: Supports user-generated content, geo-tagging, collaborative challenges, and e-commerce transactions.

### 2. Backend Layer:

Technologies Used:
   Spring Boot (Java): Handles RESTful APIs and implements core business logic.
   Python-Based Microservices: Powers AI-driven operations such as calorie adjustments and fitness recommendations.
Features Implemented:
   API Integration: Connects with wearable devices and glucose monitors to gather real-time health metrics.
   Data Processing Pipelines: Processes input data dynamically to generate personalized recommendations.

### 3. Database Layer:

Database Management System (DBMS): PostgreSQL.
Structure:
   Relational tables store:
      User profiles.
      Nutritional and fitness data.
      AI-generated recommendations and interaction history.
Optimization:
   Indexing: Enables fast query execution for real-time responsiveness.
   Partitioning: Supports efficient management of large-scale user and health data.

### 4. AI Model Deployment:

Frameworks Used:
   TensorFlow Serving: Deploys trained AI models for inference in real-time scenarios.
   Flask APIs: Facilitates communication between AI services and other system layers.
Deployment Infrastructure:
   Docker Containers: Isolate AI services for improved reliability and maintainability.
   Kubernetes Clusters: Provide scalability to handle variable computational demands.

### 5. Cloud Infrastructure:

Provider: Amazon Web Services (AWS).
Services Used:
   S3: Ensures secure storage for user-generated data, including food scans and activity logs.
   EC2: Handles scalable computational tasks for real-time processing.
   RDS (Relational Database Service): Manages PostgreSQL databases for efficient data operations.

### 6. Security Features:

End-to-End Encryption (SSL/TLS): Protects data during transmission.
Token-Based Authentication (OAuth 2.0): Ensures secure user access.
Regular Security Audits: Maintains compliance with GDPR, HIPAA, and US data protection regulations.

### Example Use Cases

### 1. Nutritional Adaptation:

A user logs their lunch using the voice command: “Run Fitzy, log my lunch of pasta and chicken.”
The AI calculates caloric and nutrient intake, updates the user's profile, and adjusts dinner recommendations to maintain the daily caloric target of 1,800 kcal.

### 2. Fitness Tracking:

A user initiates a workout session with the voice command: “Run Fitzy, track my run.”
The system tracks distance, pace, and calories burned via GPS, then recommends a recovery plan based on performance metrics.

### 3. Social Networking:

A user participates in a step challenge at their gym. Progress is tracked through geo-tagging, and the leaderboard motivates participants by offering rewards for top performances.

### 4. Food Scanning:

A user scans the barcode of a protein bar. The system extracts the nutritional information, updates the daily log, and adjusts subsequent meal recommendations to ensure a balanced caloric distribution.

1. A system for health and fitness management, comprising:
(a) a cloud-based infrastructure configured to securely store, process, and retrieve user data, including health metrics, fitness logs, and user-generated content;
(b) artificial intelligence models, including artificial neural networks, decision tree algorithms, and convolutional neural networks, trained on datasets comprising food items, nutritional values, fitness routines, user behavior, health metrics, and food images to generate personalized recommendations and analyze meal inputs;
(c) a voice recognition module configured to interpret predefined phrases and user input for logging meals, tracking workouts, recording health metrics, and executing hands-free commands;
(d) a computer vision module configured to analyze food labels and meal images to extract nutritional data, estimate portion sizes, and calculate caloric and nutrient values;
(e) an integration module configured to synchronize real-time data from wearable devices, including heart rate, activity levels, sleep patterns, and blood glucose monitors, to provide continuous health monitoring;
(f) a dynamic adjustment module configured to modify meal and workout plans in response to real-time user inputs, nutritional intake, and fitness goals, including adaptations for glycemic control;
(g) a user interface accessible via web and mobile platforms for displaying recommendations, tracking progress, visualizing trends, and enabling interactive user engagement;
(h) a social networking module configured to facilitate geo-tagged interactions, collaborative fitness challenges, content sharing, and ad revenue-sharing for user-generated content;
(i) an e-commerce module configured to facilitate transactions for health and fitness-related products, supported by artificial intelligence-driven product recommendations, price comparisons, and secure payment systems; and
(j) a security module configured to encrypt user data, ensure token-based authentication, and maintain compliance with GDPR, US data protection standards, and HIPAA.

2. A method for health and fitness management, comprising:
(a) receiving user profile data, including demographic information, health metrics, activity levels, and fitness goals;
(b) processing real-time inputs, including voice commands, food scans, and wearable device data, using artificial intelligence models trained to analyze non-linear relationships, classify user activities, and extract nutritional information;
(c) generating personalized recommendations for meal plans, fitness routines, and health monitoring based on the processed inputs, user profile data, and health metrics;
(d) dynamically adjusting meal and fitness plans in response to logged user activities, nutritional intake, glucose levels, and other updated health metrics to align with user-specific goals;
(e) enabling social connectivity by facilitating geo-tagged interactions, collaborative challenges, and content sharing, with ad revenue-sharing for user-generated content rated highly by the community;
(f) facilitating transactions for health and fitness products through an integrated e-commerce platform using artificial intelligence-driven product suggestions, price comparison algorithms, and secure payment systems; and
(g) synchronizing user data securely in real time while maintaining compliance with GDPR, HIPAA, and US data protection standards.

3. The system of claim 1, wherein the artificial neural networks are trained to analyze non-linear relationships between user health metrics, activity patterns, and fitness goals to generate adaptive recommendations.

4. The system of claim 1, wherein the voice recognition module activates with a predefined phrase and supports hands-free interactions for logging meals, tracking workouts, recording health metrics, and initiating real-time recommendations.

5. The system of claim 1, wherein the computer vision module employs convolutional neural networks trained on labeled datasets of food items and meal images to identify food components and estimate portion sizes.

6. The system of claim 1, wherein the integration module supports synchronization with external glucose monitors to track blood sugar levels and provide dietary adjustments for glycemic control.

7. The system of claim 1, wherein the social networking module includes leaderboards for collaborative fitness challenges and gamified elements, such as badges and rewards for maintaining fitness streaks.

8. The system of claim 1, wherein the e-commerce module provides real-time price comparisons for health-related products using artificial intelligence-driven analysis.

9. The system of claim 1, wherein the security module implements token-based authentication for secure user access and end-to-end encryption for all data transmissions.

10. The system of claim 1, wherein the artificial intelligence models are trained on datasets comprising nutritional profiles, fitness routines, food image libraries, user activity logs, and glycemic control data to enhance prediction accuracy.

11. The method of claim 2, wherein the meal plan recommendations are dynamically adjusted based on a user's remaining caloric target, nutrient balance, and blood glucose levels.

12. The method of claim 2, wherein food scans are analyzed using computer vision models trained on datasets of food labels, meal images, and nutritional profiles.

13. The method of claim 2, wherein fitness routines are adapted using principles of muscle confusion to ensure diverse and progressive workouts that prevent fitness plateaus.

14. The method of claim 2, wherein user recommendations are displayed via an interactive dashboard showing trends in caloric intake, fitness progress, blood glucose levels, and nutrient balance.

15. The method of claim 2, wherein transactions on the e-commerce platform are supported by artificial intelligence-driven product recommendations and secure payment gateways.

16. The method of claim 2, wherein the social networking features allow users to geo-tag activities, share fitness milestones, monetize content, and participate in location-based challenges.
